(19) United States
(12) Patent Application Publication
(10) Pub. No.: US 2021/0173362 A1
(43) Pub. Date: Jun. 10, 2021

(54) INFORMATION PROCESSING DEVICE AND NON-TRANSITORY COMPUTER READABLE MEDIUM

(71) Applicant: FUJI XEROX CO., LTD., Tokyo (JP)

(72) Inventors: Masahiro SATO, Kanagawa (JP); Kengo TOKUCHI, Kanagawa (JP)

(73) Assignee: FUJI XEROX CO., LTD., Tokyo (JP)

(21) Appl. No.: 16/840,902

(22) Filed: Apr. 6, 2020

(30) Foreign Application Priority Data
Dec. 5, 2019 (JP) 2019-220211

(51) Int. Cl. G05B 15/02; G16H 40/67; G06K 9/00

(52) U.S. Cl. CPC G05B 15/02; G06K 9/00885; G16H 40/67

(57) ABSTRACT

An information processing includes a processor. The processor is configured to output an instruction for turning equipment on or off in response to acquiring first biological information from a user that corresponds to turning the equipment on or off, and output an instruction for setting a function level of the equipment according to second biological information from the user or environmental information that corresponds to a function level of the equipment in a case where the second biological information or the environmental information is acquired.

### CROSS-REFERENCE TO RELATED APPLICATIONS

This application is based on and claims priority under 35 USC 119 from Japanese Patent Application No. 2019-220211 filed Dec. 5, 2019.

### BACKGROUND

### (i) Technical Field

The present disclosure relates to an information processing device and a non-transitory computer readable medium.

### (ii) Related Art

It is conceivable to operate equipment by using biological information such as brain waves.

Japanese Unexamined Patent Application Publication No. 2015-211705 describes a device that detects a brain-wave biological signal from the brain waves of a user, detects a surface electromyography biological signal from the surface myoelectric potential of the user, and computes a control signal on the basis of both biological signals.

Japanese Unexamined Patent Application Publication No. 2016-67922 describes a device that acquires the brain waves of the user and selectively operates multiple devices to be operated in accordance with an analysis result obtained by analyzing the brain waves.

### SUMMARY

Meanwhile, operating equipment using biological information may demand not only operations such as turning the equipment on or off, but also operations of setting a function level of the equipment.

Aspects of non-limiting embodiments of the present disclosure relate to improving the accuracy of setting a function level of equipment in a case of operating the equipment by using biological information.

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to an aspect of the present disclosure, there is provided an information processing including a processor. The processor is configured to output an instruction for turning equipment on or off in response to acquiring first biological information from a user that corresponds to turning the equipment on or off, and output an instruction for setting a function level of the equipment according to second biological information from the user or environmental information that corresponds to a function level of the equipment in a case where the second biological information or the environmental information is acquired.

### BRIEF DESCRIPTION OF THE DRAWINGS

An exemplary embodiment of the present disclosure will be described in detail based on the following figures, wherein:

FIG. 1 is a block diagram illustrating a configuration of an information processing system according to an exemplary embodiment;

FIG. 2 is a block diagram illustrating a configuration of an information processing device according to the exemplary embodiment;

FIG. 3 is a diagram illustrating a flowchart that illustrates processes by the information processing device according to the exemplary embodiment;

FIG. 4 is a diagram illustrating a flowchart that illustrates processes by the information processing device according to the exemplary embodiment;

FIG. 5 is a diagram illustrating a management table;

FIG. 6 is a diagram illustrating a management table;

FIG. 7 is a diagram illustrating a management table;

FIG. 8 is a diagram illustrating a management table;

FIG. 9 is a diagram illustrating brain waves; and

FIG. 10 is a diagram illustrating brain waves.

### DETAILED DESCRIPTION

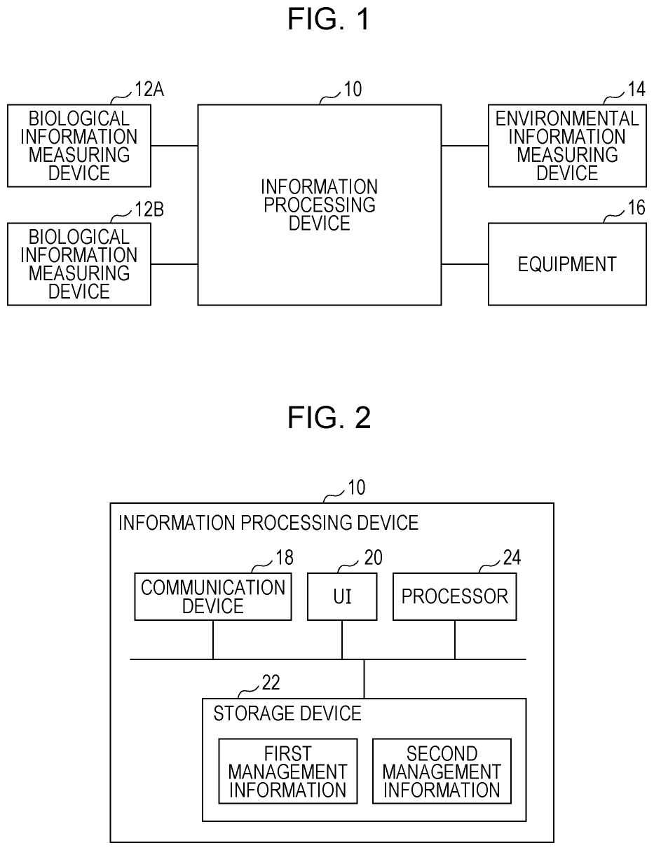

An information processing system according to the exemplary embodiment will be described with reference to FIG. 1. FIG. 1 illustrates an example of the configuration of the information processing system according to the exemplary embodiment.

The information processing system according to the exemplary embodiment includes an information processing device 10, multiple biological information measuring devices, one or more environmental information measuring devices 14, and one or more pieces of equipment 16. In the example illustrated in FIG. 1, the information processing system includes biological information measuring devices 12A and 12B, but this is merely one example. In the following, the biological information measuring devices 12A and 12B will be referred to as the “biological information measuring device(s) 12” when it is not necessary to distinguish between the biological information measuring devices 12A and 12B. Note that the number of each type of device illustrated in FIG. 1 is merely one example, and the number of each type of device is not limited to the numbers of devices illustrated in FIG. 1. In addition, the information processing system may also include other devices (for example, an external device such as a server) besides the devices illustrated in FIG. 1.

The information processing device 10, the biological information measuring device(s) 12, the environmental information measuring device(s) 14, and the equipment 16 are configured to communicate with other devices and the like. The communication may be wired communication using a cable, or wireless communication. In other words, each device may transmit and receive information with each other through physical connection to other devices and the like using a cable, or transmit and receive information with each other through wireless communication. For the wireless communication, a technology such as short-range wireless communication or Wi-Fi (registered trademark) may be used, for example. Wireless communication according to a standard other than the above may also be used. The short-range wireless communication may be a technology such as Bluetooth (registered trademark), radio-frequency identifier (RFID), or NFC, for example. Each device may also communicate with another device, another sensor, or the like through a communication channel such as a local area network (LAN) or the Internet.

The information processing device 10 is for example a personal computer (hereinafter referred to as “PC”), a tablet PC, a smartphone, a mobile phone, or some other type of device. The information processing device 10 may be a terminal device that the user is able to carry (such as a tablet PC, a smartphone, or a mobile phone, for example), or a device that is installed on a table or the like and used. The information processing device 10 may also be a smart speaker including communication functions, a microphone, and a speaker. The information processing device 10 may be installed indoors (such as on the floor, ceiling, or on a table in a room, for example), or may installed outdoors. Additionally, the information processing device 10 may also be a device capable of movement (for example, a self-propelled device).

The biological information measuring devices 12 include components such as sensors and electrodes, and are configured to measure biological information about a user. Each biological information measuring device 12 may measure a different type of biological information. Obviously, some or all of the biological information measuring devices 12 may also be configured to measure the same type of biological information. In addition, each biological information measuring device 12 may be configured to measure a single type of biological information or multiple types of biological information.

Each biological information measuring device 12 transmits biological information measured by itself to the information processing device 10. Each biological information measuring device 12 may transmit the biological information to the information processing device 10 every time the biological information is measured, or each biological information measuring device 12 may store the biological information and transmit the biological information to the information processing device 10 at predetermined time intervals or transmit the biological information to the information processing device 10 at a timing specified by the user. Also, each biological information measuring device 12 may receive biological information measured by another biological information measuring device 12 from the other biological information measuring device 12, and transmit the biological information measured by itself together with the biological information measured by the other biological information measuring device 12 to the information processing device 10.

Each biological information measuring device 12 may also analyze the biological information measured by itself or another biological information measuring device, and transmit information indicating the analysis result to the information processing device 10. For example, each biological information measuring device 12 may include a processor, and the process may analyze the biological information. Obviously, such analysis may also be performed by the information processing device 10.

The biological information measuring device 12 may include a battery and be driven by power supplied from the battery, or be driven by receiving a supply of power from the information processing device 10.

The biological information measuring device 12 may also be a wearable device that is entirely worn on the user and measures biological information. For example, the biological information measuring device 12 may be a device worn on the user's head, a bearable device worn on the user's ear, a device worn on a part of the body such as the user's arm, hand, wrist, or finger (such as a wristwatch-style device for example), a device worn around the user's neck, or a device worn on a part of the body such as the user's torso or leg.

The biological information is any of various types of physiological information and anatomical information produced by the living body of the user. As a conceptual category, biological information includes information such as information indicating the activity of the brain (such as brain waves, the quantity of cerebral blood flow, or a cerebral magnetic field signal, for example), pulse frequency, blood pressure, heart rate, an electrocardiogram waveform, a myoelectric waveform, eye movement, body temperature, perspiration, line of sight, speech, and user movement, for example. The above biological information is merely an example, and other physiological information and anatomical information may also be used as the biological information. The biological information measuring device 12 may measure one type or multiple types of biological information from among the above types of biological information.

Additionally, the conceptual category of biological information includes bioelectric potential information indicating an electric potential measured from a living body. As a conceptual category, bioelectric potential information includes information such as brain waves obtained as a result of measuring tiny electric currents produced in association with brain activity, an electrocardiogram obtained as a result of measuring tiny electric currents produced in association with the pumping action of the heart, an electromyogram obtained as a result of measuring tiny electric currents produced in association with muscle activity, and electrodermal activity obtained as a result of measuring tiny electric currents occurring in the skin, for example. The above bioelectric potential information is merely an example, and bioelectric potential information other than the above may also be used.

The information processing device 10 receives biological information from the biological information measuring device 12 and performs operations such as analyzing the biological information, storing the biological information, outputting the biological information, storing information indicating a result of analyzing the biological information, and outputting information indicating a result of analyzing the biological information. Obviously, the analysis of the biological information may also be performed by the biological information measuring device 12. Outputting the biological information means displaying the biological information or outputting the biological information as sound information, for example. Outputting information indicating a result of analyzing the biological information means displaying information indicating an analysis result or outputting an analysis result as sound information, for example. The information processing device 10 may also transmit information indicating the biological information and the analysis result to another device.

The information processing device 10 may include one or multiple biological information measuring devices 12. In other words, one or multiple biological information measuring devices 12 may be incorporated into the information processing device 10 to form a single device. The entirety of the information processing device 10 including the one or more biological information measuring devices 12 may also be worn on the user and measure biological information. In other words, the information processing device 10 may also be a wearable device. For example, the information processing device 10 may be a device worn on the user's head, a bearable device worn on the user's ear, a device worn on a part of the body such as the user's arm, hand, wrist, or finger (such as a wristwatch-style device), a device worn around the user's neck, or a device worn on a part of the body such as the user's torso or leg.

The information processing device 10 and the biological information measuring device 12 may also be separate devices. For example, the information processing device 10 may be a smart speaker, and the biological information measuring device 12 may be a wearable device worn on the user.

The environmental information measuring device 14 is configured to measure environmental information related to the environment surrounding the user and the environmental information measuring device 14. For example, the environmental information measuring device 14 is a device such as a camera that takes images, a microphone that collects sound, a temperature sensor that measures temperature, a humidity sensor that measures humidity, an odor sensor that measures odors, an illuminance sensor that measures brightness, an infrared sensor, or a presence sensor. One or more of the above sensors may be included in the information processing system as the environmental information measuring device 14.

For example, an image of the surroundings of the information processing device 10 or another place is taken by a camera, and image data expressing the surroundings or image data expressing the other place is generated. The image data may be moving image data or still image data. Image data taken by the camera corresponds to one example of environmental information related to the environment included in the image-taking range of the camera. Also, image data expressing the user that is generated by taking an image of the user with the camera corresponds to one example of biological information about the user. For example, characteristics such as user motion or the user's body type detected from the image data corresponds to one example of biological information about the user. In this sense, the camera corresponds to one example of the biological information measuring device 12 that measures biological information about the user.

Also, sounds around a microphone (for example, a person's speech and other sounds) are input into the microphone, and sound data is generated by the microphone. Sound data expressing the sound input into the microphone corresponds to one example of environmental information related to the environment surrounding the microphone. Also, sound data expressing the user's speech input into the microphone corresponds to one example of biological information about the user. In this sense, the microphone corresponds to one example of the biological information measuring device 12 that measures biological information about the user.

Likewise, data measured by sensors such as a temperature sensor, a humidity sensor, an odor sensor, an illuminance sensor, an infrared sensor, and a presence sensor corresponds to one example of environmental information. Also, data measured from the user by the above sensors corresponds to one example of biological information about the user. In this sense, the above sensors correspond to one example of the biological information measuring device 12 that measures biological information about the user.

The environmental information may also include information such as information indicating the size of the room where the user is present, information indicating the size of the room where the equipment is installed, and information indicating the number of windows provided in the room.

Note that one or multiple environmental information measuring devices 14 may be included in the information processing device 10.

The equipment 16 is a device such as a PC, a tablet PC, a smartphone, a mobile phone, a robot (such as a humanoid robot, a non-humanoid animal robot, a cleaning robot, or some other type of robot, for example), a projector, a display device such as a liquid crystal display, a recording device, a playback device, an imaging device such as a camera, a refrigerator, a rice cooker, a microwave oven, a coffee maker, a vacuum cleaner, a washing machine, an air conditioner, lighting equipment, a clock, a surveillance camera, an automobile, a motorcycle, an aircraft (such as an unmanned aerial vehicle (for example, a drone)), a game console, a gas stove, a toilet seat with a bidet function, a ventilation fan, a doorbell, an entrance monitor, an elevator, a door, a window, or any of various types of sensing equipment (such as a temperature sensor, a humidity sensor, a voltage sensor, or a current sensor), for example. Equipment in general may also be included in the conceptual category of the equipment 16. For example, information equipment, video equipment, audio equipment, and other types of equipment may be included in the category of the equipment 16 according to the exemplary embodiment.

The equipment 16 includes a communication device that acts as a communication interface, a storage device that stores data, and a processor that controls the operation of the equipment 16. The equipment 16 may also include a user interface. The equipment 16 may also transmit equipment identification information for identifying the equipment 16 itself to the information processing device 10. For example, the equipment identification information is information such as an ID, a name, a serial number, or an address (such as a MAC address or an IP address for example) of the equipment 16.

Hereinafter, FIG. 2 will be referenced to describe the configuration of the information processing device 10 in detail.

The information processing device 10 includes a communication device 18, a UI 20, a storage device 22, and a processor 24, for example. The information processing device 10 may also include components other than the above.

The communication device 18 is a communication interface, and has a function of transmitting data to other devices and a function of receiving data transmitted from other devices. The communication device 18 may have a wireless communication function, and may also have a wired communication function. The communication device 18 may communicate with another device by using short-range wireless communication for example, or communicate with another device through a communication channel such as a LAN or the Internet. For example, the communication device 18 receives biological information transmitted from the biological information measuring device 12. The communication device 18 may also transmit control information for controlling the operation of the biological information measuring device 12 to the biological information measuring device 12. In addition, the communication device 18 receives environmental information transmitted from the environmental information measuring device 14. The communication device 18 may also transmit control information for controlling the operation of the environmental information measuring device 14 to the environmental information measuring device 14. In addition, the communication device 18 transmits control information for controlling the operation of the equipment 16 to the equipment 16. The communication device 18 may also receive information transmitted from the equipment 16.

The UI 20 is a user interface, and includes a display device and an operation device. The display device is a device such as a liquid crystal display or an EL display. The operation device is a device such as a keyboard, input keys, or a control panel. The UI 20 may be a UI such as a touch panel combining a display device with an operation device. In addition, a microphone may also be included in the UI 20, and a speaker that emits sound may also be included in the UI 20.

The storage device 22 is a device that establishes one or multiple storage areas that store various types of data. For example, the storage device 22 is a hard disk drive, any of various types of memory (such as RAM, DRAM, and ROM, for example), another type of storage device (such as an optical disc, for example), or a combination of the above. One or more storage devices 22 are included in the information processing device 10.

First management information and second management information is stored in the storage device 22. The first management information and the second management information is information for specifying an operation item of the equipment 16 on the basis of the biological information measured from the user. For example, predetermined standard biological information and operation information indicating an operation item of the equipment 16 are associated with each other and registered in the first management information and the second management information in advance. The standard biological information may be biological information that is anticipated to occur in the user who performs an operation associated with the standard biological information, or biological information that is anticipated to occur in the user who requests the execution of the operation. The standard biological information may also be considered to be biological information corresponding to an operation item of the equipment 16. The standard biological information and the operation information may be associated with each other and registered in the management information for each user.

The first management information is management information in which information related to an operation item regarding the power state of the equipment 16 is registered. For example, predetermined first standard biological information and first operation information indicating a first operation item of the equipment 16 are associated with each other and registered in the first management information in advance. The first operation item is an operation of turning the equipment 16 on or off. The first operation information is information indicating an operation of turning the equipment 16 on or off. The first standard biological information may be biological information that is anticipated to occur in the user who performs the first operation item indicated by the first operation information associated with the first standard biological information, or biological information that is anticipated to occur in the user who requests the execution of the first operation item. The first standard biological information may be considered to be biological information corresponding to turning the equipment 16 on or off. The first standard biological information and the first operation information may be associated with each other and registered in the first management information for each user.

The second management information is management information in which information related to an operation item regarding a function level of the equipment 16 is registered. For example, predetermined second standard biological information and second operation information indicating a second operation item of the equipment 16 are associated with each other and registered in the second management information in advance. The second operation item is an operation of setting a function level of the equipment 16. The second operation information is information indicating a function level of the equipment 16. The second standard biological information may be biological information that is anticipated to occur in the user who performs the second operation item indicated by the second operation information associated with the second standard biological information, or biological information that is anticipated to occur in the user who requests the execution of the second operation item. The second standard biological information may be considered to be biological information corresponding to a function level of the equipment 16. The second standard biological information and the second operation information may be associated with each other and registered in the second management information for each user.

A function level is a level related to the performance or output of the equipment 16, for example. To give specific examples, the set temperature of an air conditioner, the amount of airflow of an air conditioner, the direction of airflow of an air conditioner, the activation or deactivation of a dehumidifier function of an air conditioner, the brightness of a display device, the brightness of a lighting device, the volume of a speaker, the movement speed of a self-propelled device (such as a robot or a self-propelled vacuum cleaner, for example), settings in devices such as an imaging device, a recording device, and a playback device, settings in devices such as a refrigerator, a rice cooker, and a microwave oven, settings in any of various types of sensing equipment, and the like correspond to examples of function levels. However, the above are merely an example, and set values and the like other than the above may also be function levels.

As another example of the second management information, predetermined standard environmental information and second operation information indicating a second operation item of the equipment 16 may be associated with each other and registered in the second management information in advance. The standard environmental information may be considered to be information corresponding to a function level of the equipment 16.

As yet another example of the second management information, second standard biological information, standard environmental information, and second operation information may be associated with each other and registered in the second management information in advance. The second standard biological information and the standard environmental information associated with the second operation information may be considered to be information corresponding to a function level of the equipment 16.

The first standard biological information and the second standard biological information may also be determined for each piece of equipment 16. For example, the first standard biological information and the second standard biological information may be different biological information or the same biological information for each piece of equipment 16.

The first standard biological information and the second standard biological information may be the same type of biological information or different types of biological information. For example, the first standard biological information and the second standard biological information may be biological information measured by the same biological information measuring device 12 or biological information measuring devices 12 of the same type, but may also be biological information measured by different biological information measuring devices 12 or biological information measuring devices 12 of different types.

As an example, the first standard biological information and the second standard biological information may both be brain waves. As another example, the first standard biological information may be a brain wave, and the second standard biological information may be body temperature. Obviously, the first standard biological information and the second standard biological information may also be biological information other than the above.

As yet another example, the first standard biological information and the second standard biological information may also be the same type of biological information in different frequency bands. For example, the first standard biological information may be a first brain wave in a first frequency band, while the second standard biological information may be a second brain wave in a second frequency band different from the first frequency band. The first frequency band and the second frequency band may also be partially overlapping frequency bands.

The second standard biological information may be information indicating a function level itself of the equipment 16, or information indicating an emotional or mental state of the user. Described in terms of an example of a function level of an air conditioner acting as one example of the equipment 16, the second standard biological information may be information indicating the set temperature (such as 25° C. for example) of the air conditioner, or information indicating an emotional or mental state of the user, such as feeling hot or uncomfortable.

The processor 24 is configured to acquire biological information about the user and output an instruction for operating the equipment 16 in accordance with the biological information.

For example, when biological information is measured by the biological information measuring device 12, the biological information is transmitted from the biological information measuring device 12 to the information processing device 10. The processor 24 receives the biological information and ascertains an operation item of the equipment 16 on the basis of the biological information. The processor 24 controls the operation of the equipment 16 by transmitting control information including information indicating the ascertained operation item to the equipment 16. The equipment 16 receiving the control information operates in accordance with the control information. With this arrangement, the processor 24 operates the equipment 16.

For example, in response to the acquisition of first biological information corresponding to turning the equipment 16 on or off, the processor 24 outputs an instruction for turning the equipment 16 on or off according to the first biological information to the equipment 16.

Also, in the case of acquiring second biological information corresponding to a function level of the equipment 16, the processor 24 outputs an instruction for setting a function level of the equipment 16 according to the second biological information to the equipment 16.

In the case of acquiring environmental information corresponding to a function level of the equipment 16, the processor 24 may also output an instruction for setting a function level of the equipment 16 according to the environmental information to the equipment 16.

For example, the first biological information is biological information of the same type as the first standard biological information. The second biological information is biological information of the same type as the second standard biological information.

The first biological information and the second biological information may also be determined for each piece of equipment 16. For example, the first biological information and the second biological information may be different biological information or the same biological information for each piece of equipment 16.

The first biological information and the second biological information may be the same type of biological information or different types of biological information. For example, the first biological information and the second biological information may be biological information measured by the same biological information measuring device 12 or biological information measuring devices 12 of the same type, but may also be biological information measured by different biological information measuring devices 12 or biological information measuring devices 12 of different types.

As an example, the first biological information and the second biological information may both be brain waves. As another example, the first biological information may be a brain wave, and the second biological information may be body temperature. Obviously, the first biological information and the second biological information may also be biological information other than the above.

As yet another example, the first biological information and the second biological information may also be the same type of biological information in different frequency bands. For example, the first biological information may be a first brain wave in a first frequency band, while the second biological information may be a second brain wave in a second frequency band different from the first frequency band. The first frequency band and the second frequency band may also be partially overlapping frequency bands.

The second biological information may be information indicating a function level itself of the equipment 16, or information indicating an emotional or mental state of the user. Described in terms of an example of a function level of an air conditioner acting as one example of the equipment 16, the second biological information may be information indicating the set temperature (such as 25° C. for example) of the air conditioner, or information indicating an emotional or mental state of the user, such as feeling hot or uncomfortable.

For example, in the case of acquiring first biological information corresponding to turning on the equipment 16, and acquiring second biological information corresponding to a function level of the equipment 16 within a predetermined amount of time from the point in time when the first biological information is acquired, the processor 24 may output an instruction for turning on the equipment 16 to the equipment 16, and output an instruction for setting a function level of the equipment 16 according to the second biological information to the equipment 16.

For example, a brain-machine interface may be constructed by a biological information measuring device 12 that measures the activity of the brain and the information processing device 10. The brain-machine interface may be invasive or non-invasive. In this case, the processor 24 operates the equipment 16 on the basis of the activity (such as brain waves for example) of the user's brain. To operate the equipment 16, the processor 24 may extract a characteristic component from brain waves, and operate the equipment 16 on the basis of the extracted component. To extract a characteristic component from brain waves, techniques such as a fast Fourier transform (FFT), a wavelet transform (WT), a time-frequency distribution (TFD), eigenvector methods (EM), or an autoregressive model (ARM) may be used. Also, as a method of linking brain waves to an operation item of the equipment 16 using feature vectors obtained by feature extraction, for example, techniques such as independent component analysis (ICA), k-means clustering, a support vector machine (SVM), or a convolutional neural network may be used.

In addition, the processor 24 may also be configured to receive equipment identification information transmitted from the equipment 16 and identify the equipment 16. For example, the processor 24 may transmit an acquisition request for equipment identification information to the equipment 16, and acquire equipment identification information transmitted from the equipment 16 in response to the acquisition request. Also, equipment identification information may be transmitted to the information processing device 10 from the equipment 16 made capable of communicating with the information processing device 10 by being connected to the information processing device 10 or the like, and the processor 24 may receive the equipment identification information transmitted in this way. For example, the equipment identification information is information such as information indicating a name of the equipment 16, information indicating a serial number of the equipment 16, or information indicating an address (such as a MAC address or an IP address for example) of the equipment 16.

In addition, the processor 24 may also be configured to control the operation of each unit of the information processing device 10. The processor 24 may include memory.

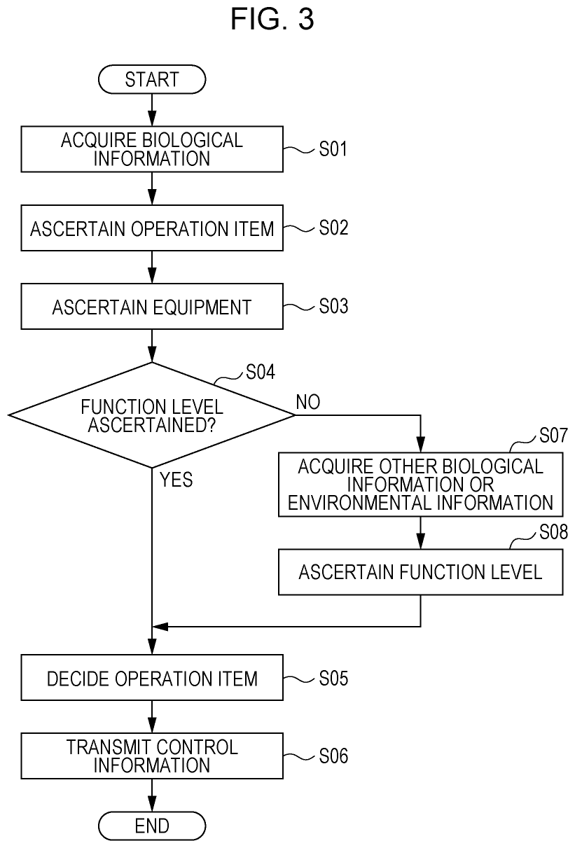

Hereinafter, FIG. 3 will be referenced to describe processes by the information processing device 10 in detail. FIG. 3 is a flowchart illustrating processes by the information processing device 10.

One or multiple pieces of biological information about the user are measured by the one or multiple biological information measuring devices 12. For example, one or multiple pieces of biological information about the user are measured by one or multiple biological information measuring devices 12 worn by the user. The biological information measured from the user is transmitted to the information processing device 10, and the processor 24 acquires the biological information (S01).

Next, the processor 24 ascertains an operation item of the equipment 16 on the basis of the biological information measured from the user (S02), and ascertains the equipment 16 to be operated (S03).

For example, the processor 24 compares biological information measured from the user to each piece of first standard biological information registered in the first management information described above, and searches for first standard biological information whose difference from the biological information measured from the user is inside a first tolerance. The processor 24 specifies the first operation item of the equipment 16 associated with the first standard biological information returned by the search. With this arrangement, the first operation item of the equipment 16 is ascertained, and the equipment 16 to be operated is ascertained. The first operation item is an operation of turning the equipment 16 on or off. Biological information whose difference from the first standard biological information is inside the first tolerance corresponds to one example of the first biological information. The first tolerance is predetermined. The first tolerance may also be changed by the user.

In the case where the search returns multiple pieces of first standard biological information whose difference from the biological information measured from the user is inside the first tolerance, the processor 24 specifies the first standard biological information having the smallest difference from the biological information measured from the user from among the multiple pieces of first standard biological information. The processor 24 specifies the first operation item of the equipment 16 associated with the specified first standard biological information.

In the case where biological information whose difference from the first standard biological information is inside the first tolerance is not measured, the first operation item (that is, the operation item of turning the equipment 16 on or off) is not specified, and the first operation item is not executed.

Also, the processor 24 compares biological information measured from the user to each piece of second standard biological information registered in the second management information described above, and searches for second standard biological information whose difference from the biological information measured from the user is inside a second tolerance. The processor 24 specifies the second operation item associated with the second standard biological information returned by the search. The second operation item is an operation of setting a function level of the equipment 16. Biological information whose difference from the second standard biological information is inside the second tolerance corresponds to one example of the second biological information. The second tolerance is predetermined. The second tolerance may also be changed by the user.

In the case where the search returns multiple pieces of second standard biological information whose difference from the biological information measured from the user is inside the second tolerance, the processor 24 specifies the second standard biological information having the smallest difference from the biological information measured from the user from among the multiple pieces of second standard biological information. The processor 24 specifies the second operation item associated with the specified second standard biological information.

In the case where biological information whose difference from the second standard biological information is inside the second tolerance is not measured, the second operation item (that is, the operation item of setting a function level of the equipment 16) is not specified, and the second operation item is not executed.

The first standard biological information may be information indicating a characteristic component of the biological information. In this case, the processor 24 may extract the characteristic component from the biological information measured from the user, and search for first standard biological information whose difference from the extracted component is inside the first tolerance.

Similarly, the second standard biological information may be information indicating a characteristic component of the biological information. In this case, the processor 24 may extract the characteristic component from the biological information measured from the user, and search for second standard biological information whose difference from the extracted component is inside the second tolerance.

For example, in the case where brain waves are used as the biological information, the processor 24 may estimate the operation item expressed by brain waves by extracting a characteristic component from measured brain waves, and analyzing the component.

On the basis of the biological information measured from the user, in the case where a function level of the equipment 16 is ascertained (S04, Yes), the processor 24 decides the operation item ascertained in S02 as the operation item of the equipment 16 (S05), and transmits control information including information indicating the operation item to the equipment 16 (S06). In this way, the processor 24 controls the operation of the equipment 16.

For example, in the case where biological information indicating an operation item of turning on a certain piece of equipment 16 (in other words, first biological information) is measured from the user, the processor 24 transmits control information including information indicating the operation item of turning on the equipment 16 to the equipment 16. With this arrangement, the equipment 16 is turned on. Also, in the case where biological information indicating an operation item of setting a function level of the equipment 16 to a certain value (in other words, second biological information) is measured from the user, the processor 24 transmits control information including information indicating the operation item of setting a function level of the equipment 16 to the value to the equipment 16. With this arrangement, the function level of the equipment 16 is set to the value.

In the case where a function level of the equipment 16 is not ascertained on the basis of the biological information measured from the user (S04, No), the processor 24 acquires other biological information measured from the user and environmental information measured by the environmental information measuring device 14 (S07), and ascertains the function level of the equipment 16 on the basis of the other biological information and the environmental information (S08). Subsequently, the process proceeds to step S05.

The other biological information is the second biological information corresponding to a function level of the equipment 16. The processor 24 searches the second management information for second standard biological information whose difference from the other biological information measured from the user is inside the second tolerance, and specifies the second operation item (in other words, a function level of the equipment 16) associated with the second standard biological information returned by the search. The other biological information may be the same type of biological information as the biological information acquired in S01, or a different type of biological information.

The processor 24 may also specify the second operation item on the basis of the measured environmental information. For example, the processor 24 searches the second management information in which standard environmental information and the second operation information are registered in association with each other for standard environmental information whose different from the measured environmental information is inside a third tolerance, and specifies the second operation item associated with the standard environmental information returned by the search. The third tolerance is predetermined. The third tolerance may also be changed by the user.

In the case where biological whose difference from the first standard biological information is inside the first tolerance is not measured, and the first operation item is not ascertained in step S02, the process ends. Also, in step S02, in the case where an operation item indicating an operation of turning off the equipment 16 is ascertained, the processor 24 turns off the equipment 16 by transmitting control information including information indicating an operation item of turning off the equipment 16 to the equipment 16, irrespectively of whether or not a function level is ascertained.

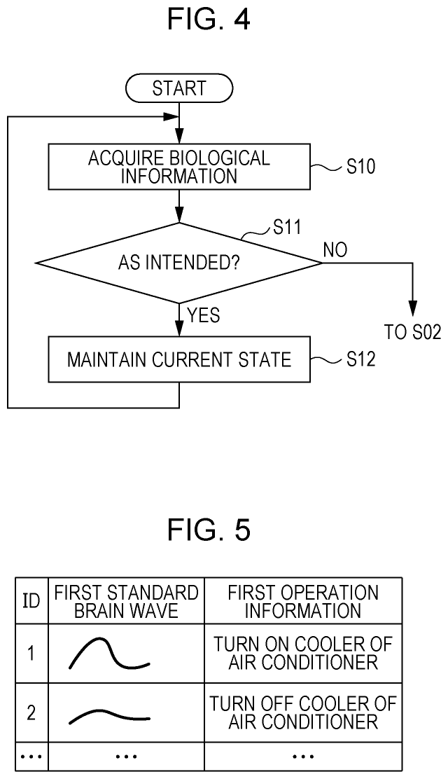

The processor 24 may also check whether or not biological information from the user acquired after the equipment 16 begins to operate in accordance with biological information is biological information conforming to the intention of the user, and check whether or not the equipment 16 is operating as intended by the user. Hereinafter, this process will be described with reference to FIG. 4. FIG. 4 is a flowchart illustrating the process.

After the equipment 16 begins to operate in accordance with biological information, the processor 24 acquires biological information measured from the user (S10).

In the case where the biological information measured from the user is biological information conforming to the intention of the user (S11, Yes), the processor 24 maintains the current operating state of the equipment 16 (S12). In other words, in the case where the equipment 16 is operating as intended by the user, the processor 24 maintains the current operating state of the equipment 16.

In the case where the biological information measured from the user is not biological information conforming to the intention of the user (S11, No), the processor 24 executes the processes from step S02. In other words, in the case where the equipment 16 is not operating as intended, the processor 24 ascertains an operation item on the basis of biological information measured from the user, and controls the operation of the equipment 16 by transmitting control information including information indicating the operation item to the equipment 16.

Hereinafter, the process of checking whether or not biological information from the user is biological information conforming to the intention of the user will be described in detail.

For example, the processor 24 ascertains an emotional or mental state of the user on the basis of biological information measured from the user before operating the equipment 16 in accordance with biological information, and sets an intention corresponding to the emotional or mental state as the intention of the user. The processor 24 may also set the intention of the user on the basis of the biological information acquired in step S01.

For example, in the case where the biological information measured from the user before the equipment 16 is operated expresses an emotion indicating discomfort due to a high room temperature, the processor 24 sets lowering the room temperature as the intention of the user. In the case where biological information measured after the equipment 16 operates expresses an emotion indicating slight comfort due to the room temperature falling, or in other words, in the case where the equipment 16 is operating to achieve the intention of the user, the processor 24 determines that the biological information from the user is biological information conforming to the intention of the user. To put it another way, in the case where a problem or an uncomfortable emotion experienced by the user is being addressed, the biological information from the user is determined to be biological information conforming to the intention of the user. In this case, the processor 24 maintains the current operating state of the equipment 16.

On the other hand, in the case where biological information measured after the equipment 16 operates expresses an emotion indicating discomfort due to a high room temperature, or in other words, in the case where the equipment 16 is not operating to achieve the intention of the user, the processor 24 determines that the biological information from the user is not biological information conforming to the intention of the user. In this case, the processor 24 executes the process from step S02.

Also, in the case where the biological information measured after the equipment 16 (hereinafter referred to as the “first equipment 16” for convenience) operates is not biological information conforming to the intention of the user, the processor 24 may also cause another piece of equipment 16 (hereinafter referred to as the “second equipment 16” for convenience) to operate in cooperation with the first equipment 16. For example, the second equipment 16 is equipment that exhibits an effect similar to the first equipment 16. The second equipment 16 may be the same type of equipment as the first equipment 16, or a different type of equipment from the first equipment 16. For example, in the case where an air conditioner is used as the first equipment 16 for lowering the room temperature, the second equipment 16 may be equipment having a function of lowering the room temperature, equipment having a function of acting on the air, or the like. For example, the second equipment 16 may be an air conditioner or a fan. The processor 24 may lower the room temperature by operating multiple air conditioners or lower the room temperature by operating an air conditioner and a fan.

The processor 24 may also decide the second equipment 16 according to a predetermined priority. For example, the priority of the second equipment 16 is decided in order of the highest anticipated effect when combined with the first equipment 16 operated in accordance with biological information. For example, in the case where the first equipment 16 being operated is an air conditioner and the operation item of the air conditioner is cooling, another air condition is given first priority, while a fan is given second priority. On the other hand, in the case where the equipment 16 being operated is an air conditioner and the operation item of the air conditioner is heating, another air condition is given first priority, while another heating appliance such as a heater is given second priority.

For example, the processor 24 decides the equipment 16 having first priority as the second equipment 16, and operates the equipment 16 having first priority. If biological information conforming to the intention of the user still is not measured in this case, the processor 24 additionally decides the equipment 16 having second priority as the second equipment 16, and operates the equipment 16 having second priority. In other words, the processor 24 operates the first equipment 16 and the two pieces of second equipment 16. The process thereafter is similar.

As another example, if biological information conforming to the intention of the user still is not measured in the case where the equipment 16 having first priority is operated, the processor 24 may also operate the equipment 16 having second priority instead of the equipment 16 having first priority. In this case, the processor 24 turns off the equipment 16 having first priority. The process thereafter is similar.

Note that the processor 24 may also notify the user about operating the second equipment 16 before operating the second equipment 16. For example, the processor 24 may cause information indicating the operation of the second equipment 16 to be displayed on the UI 20, or cause information indicating the same to be emitted as sound. In the case where the user allows the operation of the second equipment 16, the processor 24 operates the second equipment 16. In the case where the user does not allow the operation of the second equipment 16, the processor 24 does not operate the second equipment 16.

The processor 24 may also set the intention of the user on the basis of behavior by the user and biological information from the user. The processor 24 specifies the behavior of the user on the basis of information such as image data measured by the environmental information measuring device 14. For example, in the case where biological information measured from the user before the equipment 16 is operated expresses an emotion indicating discomfort due to a high room temperature, the processor 24 sets a different intention for each of the case where an amount of movement by the user is equal to or greater than a threshold and the case where the amount of movement is less than the threshold. For example, in the case where the amount of movement by the user is equal to or greater than the threshold (such as in the case where the user is running, for example), the processor 24 sets lowering the room temperature further compared to the case where the amount of movement by the user is less than the threshold as the intention of the user.

Additionally, the processor 24 may also evaluate the effect of the operation of the equipment 16 on the basis of an amount of change in biological information from the user. The amount of change in biological information is the difference between biological information measured from the user before the equipment 16 operates and biological information measured from the user after the equipment 16 operates. For example, in the case where a brain wave measured from the user before the equipment 16 operates expresses an uncomfortable emotion and a brain wave measured from the user after the equipment 16 operates expresses a relaxed emotion, the processor 24 evaluates the current operating state of the equipment 16 as effective, and maintains the current operating state of the equipment 16. For example, in the case where the type of brain waves referred to as alpha waves increase after the equipment 16 operates compared to before the equipment 16 operates, the processor 24 determines that the user is relaxed. On the other hand, in the case where the alpha waves decrease after the equipment 16 operates compared to before the equipment 16 operates, the processor 24 determines that the user is not relaxed, and executes the process from step S02.

Hereinafter, a specific example will be given to describe the information processing system according to the exemplary embodiment in detail.

FIG. 5 illustrates an example of a first management table as one example of the first management information. The data in the first management table is stored in the storage device 22. The data in the first management table may also be stored in an external device such as a server, without being stored in the storage device 22.

In the first management table, an ID, a first standard brain wave, and first operation information indicating a first operation item of the equipment 16 are associated with each other in advance. The first standard brain wave is one example of the first standard biological information. Herein, a brain wave is used as one example of the first standard biological information, but biological information other than brain waves may also be used as the first standard biological information.

The ID is information for managing the information registered in the first management table.

The first standard brain wave is determined by statistical processing, for example, and is a brain wave that is generally anticipated to be occur in the user who performs the first operation item associated with the first standard brain wave, or a brain wave that is generally anticipated to occur in the user who requests the execution of the first operation item.

The first standard brain wave may be a brain wave in a specific frequency band, or a brain wave containing brain waves in multiple frequency bands.

The first operation information is information including equipment identification information for identifying the equipment 16 to be operated and information indicating the first operation item to be performed with respect to the equipment 16. The first operation item is an operation of turning the equipment 16 on or off. The first standard brain wave is a brain wave expressing the turning on or off of the equipment 16.

For example, the first standard brain wave associated with the ID “1” is a brain wave expressing an operation item of turning on the cooler of an air conditioner. The first standard brain wave associated with the ID “2” is a brain wave expressing an operation item of turning off the cooler of an air conditioner.

For example, in the case where a brain wave whose difference from the first standard brain wave associated with the ID “1” is inside the first tolerance is measured from the user, the processor 24 specifies the first operation item of “turn on cooler of air conditioner” associated with the first standard brain wave. Additionally, the processor 24 transmits control information including information indicating the first operation item of “turn on cooler of air conditioner” to the air conditioner. The air conditioner operates in accordance with the control information. With this arrangement, the cooler of the air conditioner turns on.

Also, the processor 24 may compute a similarity between a brain wave measured from the user and a first standard brain wave, and determine whether or not the similarity is a threshold or greater. The threshold is a value corresponding to the tolerance. In the case where the similarity between the brain wave measured from the user and the first standard brain wave is the threshold or greater, the processor 24 determines that the brain wave measured from the user and the first standard brain wave are similar to each other. In other words, the processor 24 determines that the difference between the brain wave measured from the user and the first standard brain wave is inside the tolerance. In the case where a brain wave whose similarity with the first standard brain wave associated with the ID “1” is the threshold or greater is measured from the user, the processor 24 specifies the first operation item of “turn on cooler of air conditioner”.

The first standard biological information and the first operation information may be associated with each other and registered in the first management table for each user. For example, biological information measured from a user may be registered as first standard biological information for the user in the first management table.

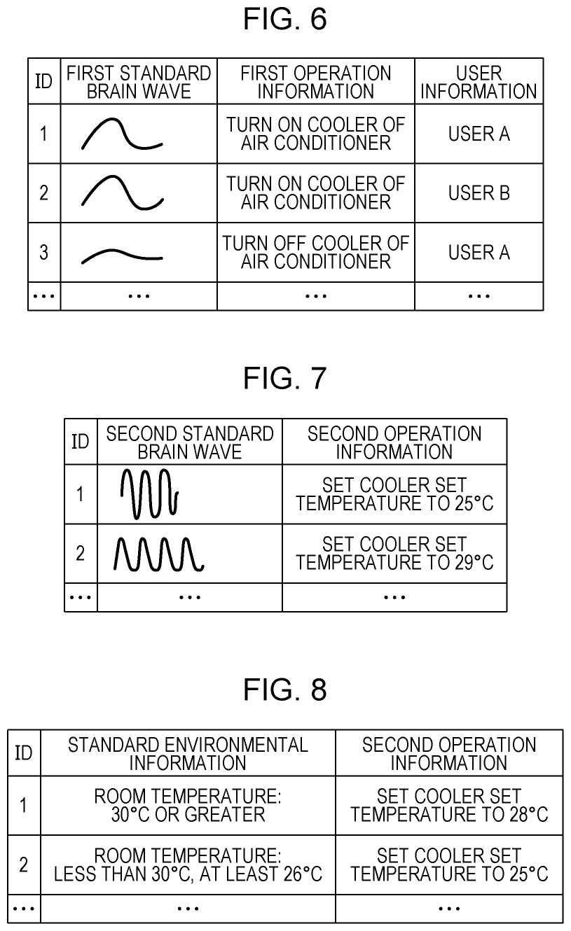

FIG. 6 illustrates an example of a first management table in which specific first standard biological information for individual users is registered. In the management table illustrated in FIG. 6, an ID, a first standard brain wave given as an example of first standard biological information, first operation information, and user information are associated with each other. The user information is information for identifying each user (such as a username or a user ID, for example).

The first standard brain wave associated with the user information is a brain wave measured from the user when the user indicated by the user information performs the first operation item associated with the first standard brain wave, or a brain wave measured from the user when the user requests the first operation item. Each first standard brain wave measured from each user is measured from each user in advance and registered in the first management table.

For example, the brain wave of a user A when the user A manually turns on the cooler of the “air conditioner” is measured by the biological information measuring device 12, and the measured brain wave is registered in the first management table as a first standard brain wave expressing the first operation item of “turn on cooler of air conditioner” by the user A.

In this case, the measured first standard brain wave of the user A is registered in the first management table in association with first operation information indicating the first operation item of “turn on cooler of air conditioner” and user information for identifying the user A. The registration may be performed using the information processing device 10 or using another device. In the example illustrated in FIG. 6, the information is registered as the information with the ID “1”. The same applies to other operations and other users.

Note that the work of registration may also be performed multiple times, and the average of multiple brain waves measured thereby may also be registered as a first standard brain wave. For example, the work of the user manually turning on the cooler of the “air conditioner” and the biological information measuring device 12 measuring the brain wave produced from the user A at the time may be performed multiple times, and an average of the multiple brain waves measured thereby may be registered in a management table as a first standard brain wave of the user A.

For example, in the case where the user A is logged in to the information processing device 10, and a brain wave whose difference from the first standard brain wave associated with the ID “1” is inside the first tolerance is measured from the user A, the processor 24 turns on the cooler of the “air conditioner” by transmitting control information including the first operation information associated with the ID “1” to the “air conditioner”. Described in further detail, if a brain wave is measured by the biological information measuring device 12 while the user A is logged in to the information processing device 10, the processor 24 searches for a first standard brain wave that is registered in the first management table in association with user information for identifying the logged-in user A. In the example illustrated in FIG. 6, the first standard brain wave associated with the ID “1” and the first standard brain wave associated with the ID “3” are registered in the first management table as first standard brain waves of the user A, and therefore these first standard brain waves are returned by the search. In the case where the difference between the measured brain wave and the first standard brain wave associated with the ID “1” is inside the first tolerance, the processor 24 turns on the cooler of the “air conditioner” by transmitting control information including the first operation information associated with the ID “1” to the “air conditioner”. In the case where the difference between the measured brain wave and the first standard brain wave associated with the ID “3” is inside the first tolerance, the processor 24 turns off the cooler of the “air conditioner” by transmitting control information including the first operation information associated with the ID “3” to the “air conditioner”.

As another example, in the case where the user operating the equipment 16 is set to “user A” in the information processing device 10, and the first standard brain wave associated with the ID “1” is measured from the user A, the processor 24 may turn on the cooler of the “air conditioner” by transmitting control information including the first operation information associated with the ID “1” to the “air conditioner”. Described in further detail, if a brain wave is measured by the biological information measuring device 12 while the user operating the equipment 16 is set to “user A” in the information processing device 10, the processor 24 searches for a first standard brain wave that is registered in the first management table in association with user information for identifying the user A who is the user operating the equipment 16. In the case where the difference between the measured brain wave and the first standard brain wave associated with the ID “1” is inside the first tolerance, the processor 24 turns on the cooler of the “air conditioner” by transmitting control information including the first operation information associated with the ID “1” to the “air conditioner”. The user operating the equipment 16 is set in the information processing device 10 by the user, for example.

For users other than the user A, each piece of information is likewise registered in the first management table similarly to the user A. For example, each piece of information associated with the ID “2” is information related to an operation when a user B turns on the cooler of the “air conditioner”. Each piece of information associated with the ID “3” is information related to an operation when the user A turns off the cooler of the “air conditioner”.

FIG. 7 illustrates an example of a second management table as one example of the second management information. The data in the second management table is stored in the storage device 22. The data in the second management table may also be stored in an external device such as a server, without being stored in the storage device 22.

In the second management table, an ID, a second standard brain wave, and second operation information indicating a second operation item of the equipment 16 are associated with each other in advance. The second standard brain wave is one example of the second standard biological information. Herein, a brain wave is used as one example of the second standard biological information, but biological information other than brain waves may also be used as the second standard biological information.

The ID is information for managing the information registered in the second management table.

The second standard brain wave is determined by statistical processing, for example, and is a brain wave that is generally anticipated to be occur in the user who performs the second operation item associated with the second standard brain wave, or a brain wave that is generally anticipated to occur in the user who requests the execution of the second operation item.

The second standard brain wave may be a brain wave in a specific frequency band, or a brain wave containing brain waves in multiple frequency bands. The first standard brain wave and the second standard brain wave may be brain waves in different frequency bands, or brain waves in the same frequency band.

The second operation information is information including equipment identification information for identifying the equipment 16 to be operated and information indicating the second operation item to be performed with respect to the equipment 16. The second operation item is an operation of setting a function level of the equipment 16. The second standard brain wave is a brain wave expressing a function level of the equipment 16.

In the example illustrated in FIG. 7, examples of function levels of an air conditioner acting as one example of the equipment 16 are illustrated. For example, properties such as the set temperature, the amount of airflow, the direction of airflow, and the presence or absence of a dehumidifier function of the air conditioner are registered in the second management table as an example of function levels of the air conditioner.

For example, the second standard brain wave associated with the ID “1” is a brain wave expressing an operation item of “set cooler set temperature of air conditioner to 25° C.”. The second standard brain wave associated with the ID “2” is a brain wave expressing an operation item of “set cooler set temperature of air conditioner to 29° C.”.

For example, in the case where a brain wave whose difference from the second standard brain wave associated with the ID “1” is inside the second tolerance is measured from the user, the processor 24 specifies the second operation item of “set cooler set temperature of air conditioner to 25° C.” associated with the second standard brain wave. Additionally, the processor 24 transmits control information including information indicating the second operation item of “set cooler set temperature of air conditioner to 25° C.” to the air conditioner. The air conditioner operates in accordance with the control information. With this arrangement, the set temperature of the cooler of the air conditioner is set to 25° C.

Also, the processor 24 may compute a similarity between a brain wave measured from the user and a second standard brain wave, and determine whether or not the similarity is a threshold or greater. The threshold is a value corresponding to the tolerance. In the case where the similarity between the brain wave measured from the user and the second standard brain wave is the threshold or greater, the processor 24 determines that the brain wave measured from the user and the second standard brain wave are similar to each other. In other words, the processor 24 determines that the difference between the brain wave measured from the user and the second standard brain wave is inside the second tolerance. In the case where a brain wave whose similarity with the second standard brain wave associated with the ID “1” is the threshold or greater is measured from the user, the processor 24 specifies the second operation item of “set cooler set temperature of air conditioner to 25° C.”.

Like the first management table illustrated in FIG. 6, the second standard biological information and the second operation information may be associated with each other and registered in the second management table for each user. For example, biological information measured from a user may be registered as second standard biological information for the user in the second management table.

FIG. 8 illustrates another example of the second management table.

In the second management table illustrated in FIG. 8, an ID, standard environmental information, and second operation information indicating a second operation item of the equipment 16 are associated with each other in advance.

For example, the standard environmental information associated with the ID “1” is information indicating that the room temperature is 30° C. or greater. The standard environmental information associated with the ID “2” is information indicating that the room temperature is 26° C. or greater and also less than 30° C.

For example, in the case where the temperature measured by a temperature sensor acting as the environmental information measuring device 14 is 30° C. or greater, the processor 24 specifies the second operation item of “set cooler set temperature of air conditioner to 28° C.” associated with the ID “1”. Additionally, the processor 24 transmits control information including information indicating the second operation item of “set cooler set temperature of air conditioner to 28° C.” to the air conditioner. The air conditioner operates in accordance with the control information. With this arrangement, the set temperature of the cooler of the air conditioner is set to 28° C.

As yet another example of the second management table, an ID, a second standard brain wave, standard environmental information, and second operation information may be associated with each other and registered in the second management table in advance. In this case, if an environmental condition indicated by the standard environmental information is satisfied and a brain wave whose difference from the second standard brain wave associated with the standard environmental information is inside the second tolerance is measured from the user, the processor 24 specifies the second operation item associated with the second standard brain wave. The processor 24 transmits control information including the second operation information indicating the second operation item to the equipment 16.

Note that the second standard brain wave may also be a brain wave indicating an emotional or mental state of the user, such as feeling hot or uncomfortable. Also, a result of analyzing the second standard brain wave may also be used instead of the second standard brain wave.

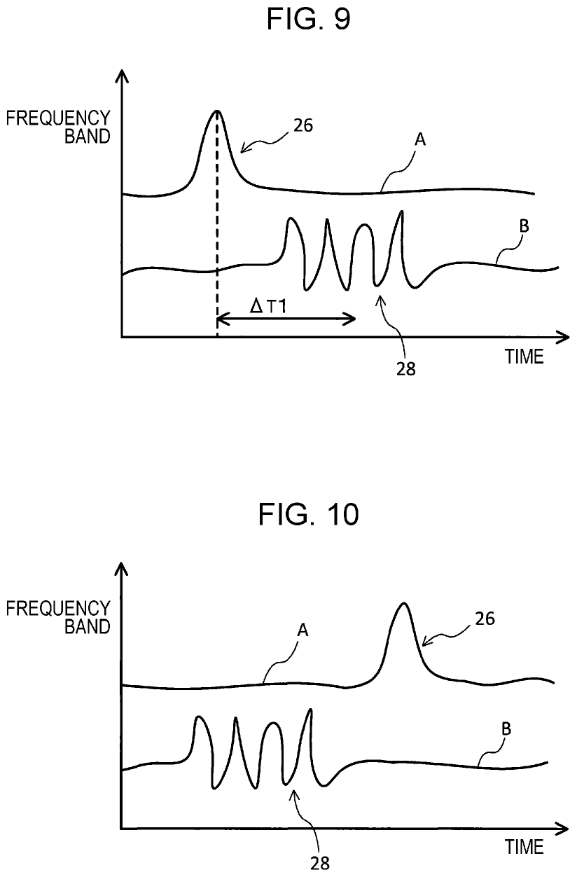

Hereinafter, FIG. 9 will be referenced to describe a specific example of operations by the information processing device 10. FIG. 9 illustrates brain waves as one example of biological information. The horizontal axis represents time, while the vertical axis represents the frequency of the brain waves. The brain waves illustrated in FIG. 9 are expressed schematically. Herein, as an example, brain waves are used as the biological information.

For example, the first standard brain wave and the second standard brain wave are brains in different frequency bands. The brain wave A is a brain wave in the same frequency band as the first standard brain wave. The brain wave B is a brain wave in the same frequency band as the second standard brain wave. The brain waves A and b are measured by the biological information measuring device 12. The processor 24 acquires the brain waves A and B measured by the biological information measuring device 12.

The processor 24 determines whether or not the brain wave A corresponding to the first operation item is measured from the user by comparing the brain wave A to the first standard brain wave registered in the first management table illustrated in FIG. 5 or 6.

The processor 24 also determines whether or not the brain wave B corresponding to the second operation item is measured from the user by comparing the brain wave B to the second standard brain wave registered in the second management table illustrated in FIG. 7.

A waveform 26 in the brain wave A is a waveform whose difference from the first standard brain wave associated with the ID “1” in FIG. 5 is inside the first tolerance. When the waveform 26 is measured, the processor 24 specifies the first operation item of “turn on cooler of air conditioner” associated with the ID “1”.

A waveform 28 in the brain wave B is a waveform whose difference from the second standard brain wave associated with the ID “1” in FIG. 7 is inside the second tolerance. When the waveform 28 is measured, the processor 24 specifies the second operation item of “set cooler set temperature of air conditioner to 25° C.” associated with the ID “1”.

In the case where the waveform 28 is measured within a predetermined amount of time ΔT1 from the point in time when the waveform 26 is measured, the processor 24 transmits control information including information indicating the first operation item of “turn on cooler of air conditioner” and information indicating the second operation item of “set cooler set temperature of air conditioner to 25° C.” to the air conditioner. With this arrangement, the cooler of the air conditioner is turned on, and the set temperature of the cooler is set to 25° C. The amount of time ΔT1 may also be changed by the user.

The point in time when the waveform 26 is measured may be the point in time when the peak of the waveform 26 is measured or the point in time when an amplitude equal to or greater than a threshold is measured in the waveform 26. The same applies to the point in time when the waveform 28 is measured.

In the case where the brain wave B whose difference from the second standard brain wave is inside the second tolerance is not measured within the amount of time ΔT1 from the point in time when the waveform 26 is measured, the processor 24 may transmit control information including information indicating the first operation item of “turn on cooler of air conditioner” to the air conditioner in accordance with the waveform 26, but the processor 24 does not have to transmit control information including information indicating the first operation item to the air conditioner.

In the case where the waveform 26 corresponding to the first operation item is not measured, but the waveform 28 is measured, the processor 24 does not transmit control information to the air conditioner. Obviously, even in this case, the processor 24 may also operate the air conditioner by transmitting control information including information indicating the second operation item corresponding to the waveform 28 to the air conditioner.

The brain wave B may be a brain wave expressing an emotional or mental state, such as feeling hot or uncomfortable. The processor 24 may also transmit control information including information indicating a second operation item of setting a function level according to the degree of emotion to the equipment 16.

For example, in the case where the waveform 26 in the brain wave A expresses a first operation item of turning on the cooler of the air conditioner, the processor 24 determines that the degree of emotion felt by the user corresponds to the set temperature of the cooler. For example, the higher the degree of discomfort, the lower the processor 24 sets the set temperature of the cooler. The processor 24 transmits control information including information indicating the second operation item of setting the set temperature according to the degree of discomfort to the air conditioner.

As another example, in the case where the waveform 26 in the brain wave A expresses a first operation item of turning on a heater of the air conditioner, the processor 24 determines that the degree of emotion felt by the user corresponds to the set temperature of the heater. For example, the higher the degree of discomfort, the higher the processor 24 sets the set temperature of the heater.

Biological information other than brain waves may also be used. For example, information indicating body temperature may also be used as the second biological information. In this case, the processor 24 transmits control information including information indicating a second operation item of setting a function level according to the body temperature to the equipment 16.

For example, in the case where the waveform 26 in the brain wave A expresses a first operation item of turning on the cooler of the air conditioner, the processor 24 determines that the body temperature of the user corresponds to the set temperature of the cooler. For example, the higher the body temperature, the lower the processor 24 sets the set temperature of the cooler. The processor 24 transmits control information including information indicating the second operation item of setting the set temperature according to the body temperature to the air conditioner.

As another example, in the case where the waveform 26 in the brain wave A expresses a first operation item of turning on the heater of the air conditioner, the processor 24 determines that the body temperature of the user corresponds to the set temperature of the heater. For example, the lower the body temperature, the higher the processor 24 sets the set temperature of the heater.

Environmental information may also be used instead of the second biological information. For example, in the case where the waveform 26 in the brain wave A expresses a first operation item of turning on the cooler of the air conditioner, the processor 24 determines that the room temperature corresponds to the set temperature of the cooler. For example, the processor 24 references the second management table illustrated in FIG. 8 to specify the second operation item corresponding to the room temperature, and transmit control information including information indicating the second operation item to the air conditioner.

Also, information indicating the size of the room where the equipment 16 is installed and information indicating the size of the room where the user is present may also be used as the environmental information. For example, an imaging device such as a camera may take an image of the room, and the processor 24 may measure the size of the room on the basis of image data generated by the imaging. As another example, the size of the room may also be measured by a radar rangefinder.

For example, in the case where the waveform 26 in the brain wave A expresses a first operation item of turning on the cooler of the air conditioner, the processor 24 determines that the size of the room corresponds to the set temperature of the cooler. For example, the larger the room, the lower the processor 24 sets the set temperature of the cooler. The processor 24 transmits control information including information indicating the second operation item of setting the set temperature according to the size of the room to the air conditioner.

As another example, in the case where the waveform 26 of the brain wave A expresses a first operation item of turning on the heater of the air conditioner, the processor 24 determines that the size of the room corresponds to the set temperature of the heater. For example, the larger the room, the higher the processor 24 sets the set temperature of the heater.

As yet another example, information indicating the number of windows provided in the room may also be used as the environmental information. For example, an imaging device such as a camera may take an image of the room, and the processor 24 may measure the number of windows provided in the room on the basis of image data generated by the imaging.

For example, in the case where the waveform 26 in the brain wave A expresses a first operation item of turning on the cooler of the air conditioner, the processor 24 determines that the number of windows corresponds to the set temperature of the cooler. For example, the higher the number of windows, the lower the processor 24 sets the set temperature of the cooler. The processor 24 transmits control information including information indicating the second operation item of setting the set temperature according to the number of windows to the air conditioner.

As another example, in the case where the waveform 26 of the brain wave A expresses a first operation item of turning on the heater of the air conditioner, the processor 24 determines that the number of windows corresponds to the set temperature of the heater. For example, the higher the number of windows, the higher the processor 24 sets the set temperature of the heater.

Also, in the case where the waveform 28 is measured within the amount of time ΔT1 from the point in time when the waveform 26 is measured, and the waveform 28 continues to be measured over an amount of time T2, the processor 24 may transmit control information including information indicating the first operation item and information indicating the second operation item to the equipment 16. Even in the case where the waveform 28 is measured within the amount of time ΔT1, if the waveform 28 does not continue to be measured for the amount of time T2 or longer, the processor 24 does not have to transmit control information including information indicating the second operation item to the equipment 16. In this case, the processor 24 may transmit control information including information indicating the first operation item to the equipment 16, but the processor 24 does not have to transmit the control information to the equipment 16.

Also, in the case where a number of waveforms 26 equal to or greater than a predetermined number are measured within a predetermined amount of time, the processor 24 may determine to cause the equipment 16 to execute the first operation item. Similarly, in the case where a number of waveforms 28 equal to or greater than a predetermined number are measured within a predetermined amount of time, the processor 24 may determine to cause the equipment 16 to execute the second operation item.

FIG. 10 illustrates another specific example. In the case where the waveform 26 is measured within the amount of time ΔT1 from the point in time when the waveform 28 is measured, the processor 24 may likewise transmit control information including information indicating the first operation item of “turn on cooler of air conditioner” and information indicating the second operation item of “set cooler set temperature of air conditioner to 25° C.” to the air conditioner. In other words, the processor 24 may operate the air conditioner even in the case where the waveform 28 is measured before the waveform 26.

In the case where the brain wave A whose difference from the first standard brain wave is inside the first tolerance is not measured within the amount of time ΔT1 from the point in time when the waveform 28 is measured, the processor 24 does not transmit control information to the air conditioner. Obviously, in this case, the processor 24 may likewise operate the air conditioner by transmitting control information including information indicating the second operation item corresponding to the waveform 28 to the air conditioner.

Note that a combination of multiple pieces of biological information may be used as the first biological information to specify the first operation item, a combination of multiple pieces of biological information may be used as the second biological information to specify the second operation item, a combination of multiple pieces of environmental information may be used to specify the second operation item, and a combination of biological information and environmental information may be used to specify the second operation item.

In the embodiment above, the term “processor” refers to hardware in a broad sense. Examples of the processor includes general processors (e.g., CPU: Central Processing Unit), dedicated processors (e.g., GPU: Graphics Processing Unit, ASIC: Application Integrated Circuit, FPGA: Field Programmable Gate Array, and programmable logic device).

In the embodiment above, the term “processor” is broad enough to encompass one processor or plural processors in collaboration which are located physically apart from each other but may work cooperatively. The order of operations of the processor is not limited to one described in the embodiment above, and may be changed.

The foregoing description of the exemplary embodiment of the present disclosure has been provided for the purposes of illustration and description. It is not intended to be exhaustive or to limit the disclosure to the precise forms disclosed. Obviously, many modifications and variations will be apparent to practitioners skilled in the art. The embodiment was chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing device comprising:
a processor configured to
output an instruction for turning equipment on or off in response to acquiring first biological information from a user that corresponds to turning the equipment on or off, and
output an instruction for setting a function level of the equipment according to second biological information from the user or environmental information that corresponds to a function level of the equipment in a case where the second biological information or the environmental information is acquired.

2. The information processing device according to claim 1, wherein
the processor is configured to
output an instruction for turning on the equipment and output an instruction for setting a function level of the equipment according to the second biological information in a case where the second biological information is acquired within a predetermined amount of time from a point in time when the first biological information that corresponds to turning on the equipment is acquired.

3. The information processing device according to claim 1, wherein
the first biological information and the second biological information are a same type of biological information in different frequency bands.

4. The information processing device according to claim 2, wherein
the first biological information and the second biological information are a same type of biological information in different frequency bands.

5. The information processing device according to claim 1, wherein
the processor is additionally configured to
confirm whether or not the biological information from the user acquired after the equipment begins to operate is biological information conforming to an intention of the user.

6. The information processing device according to claim 2, wherein
the processor is additionally configured to
confirm whether or not the biological information from the user acquired after the equipment begins to operate is biological information conforming to an intention of the user.

7. The information processing device according to claim 3, wherein
the processor is additionally configured to
confirm whether or not the biological information from the user acquired after the equipment begins to operate is biological information conforming to an intention of the user.

8. The information processing device according to claim 4, wherein
the processor is additionally configured to
confirm whether or not the biological information from the user acquired after the equipment begins to operate is biological information conforming to an intention of the user.

9. The information processing device according to claim 5, wherein
the processor is additionally configured to
cause the equipment to operate in coordination with other equipment according to a result of the confirmation.

10. The information processing device according to claim 6, wherein
the processor is additionally configured to
cause the equipment to operate in coordination with other equipment according to a result of the confirmation.

11. The information processing device according to claim 9, wherein
the other equipment is decided according to a priority.

12. The information processing device according to claim 10, wherein
the other equipment is decided according to a priority.

13. The information processing device according to claim 5, wherein
the intention of the user is determined on a basis of behavior by the user and the biological information from the user.

14. The information processing device according to claim 9, wherein
the intention of the user is determined on a basis of behavior by the user and the biological information from the user.

15. The information processing device according to claim 11, wherein
the intention of the user is determined on a basis of behavior by the user and the biological information from the user.

16. The information processing device according to claim 1, wherein
the processor is additionally configured to
evaluate an effect of the operation of the equipment on a basis of an amount of change in the biological information from the user.

17. The information processing device according to claim 2, wherein
the processor is additionally configured to
evaluate an effect of the operation of the equipment on a basis of an amount of change in the biological information from the user.

18. The information processing device according to claim 3, wherein
the processor is additionally configured to
evaluate an effect of the operation of the equipment on a basis of an amount of change in the biological information from the user.

19. A non-transitory computer readable medium storing a program causing a computer to execute a process for processing information, the process comprising:
outputting an instruction for turning equipment on or off in response to acquiring first biological information from a user that corresponds to turning the equipment on or off, and
outputting an instruction for setting a function level of the equipment according to second biological information from the user or environmental information that corresponds to a function level of the equipment in a case where the second biological information or the environmental information is acquired.

20. An information processing device comprising:
power instruction outputting means for outputting an instruction for turning equipment on or off in response to acquiring first biological information from a user that corresponds to turning the equipment on or off; and
function level instruction outputting means for outputting an instruction for setting a function level of the equipment according to second biological information from the user or environmental information that corresponds to a function level of the equipment in a case where the second biological information or the environmental information is acquired.
